(19) United States
(12) Patent Application Publication
(10) Pub. No.: US 2011/0288294 A1
(43) Pub. Date: Nov. 24, 2011

(54) Preparation process for an inhibitor of a blood clotting factor

(76) Inventors: Michael Nonnenmacher, Leimen (DE); Joerg Jung, Floersheim (DE)

(21) Appl. No.: 13/107,879

(22) Filed: May 14, 2011

(30) Foreign Application Priority Data
May 21, 2010 (DE) 10 2010 021 188.5

(51) Int. Cl. C07D 413/14

(52) U.S. Cl. 544/137

(57) ABSTRACT

The invention relates to a process for the preparation of 5-chloro-N-({(5S)-2-oxo-3-[4-(3-oxo-4-morpholinyl)phenyl]-1,3-oxazolidin-5-yl}methyl)-2-thiophenecarboxamide (I) from 5-chlorothiophene-2-carboxylic acid and 4-[4-((S)-5-aminomethyl-2-oxooxazolidin-3-yl)phenyl]morpholin-3-one (II)

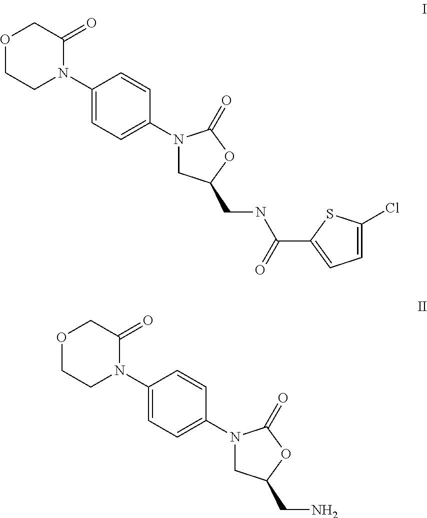

or a salt of this compound with a mineral acid or an organic acid in the presence of propanephosphonic anhydride.

### CROSS-REFERENCE TO RELATED APPLICATIONS

This application claims priority to German Patent Application 10 2010 021 188.5 filed May 21, 2010 which is hereby incorporated herein by reference in its entirety.

### FIELD OF THE INVENTION

The present invention relates to a process for the preparation of 5-chloro-N-({(5S)-2-oxo-3-[4-(3-oxo-4-morpholinyl)phenyl]-1,3-oxazolidin-5-yl}methyl)-2-thiophenecarboxamide (I) from 5-chlorothiophene-2-carboxylic acid and 4-[4-((S)-4-aminomethyl-2-oxoimidazolidin-1-yl)phenyl]morpholin-3-one (II) and salts of this compound. The target compound can be used as inhibitor of the blood clotting factor Xa.

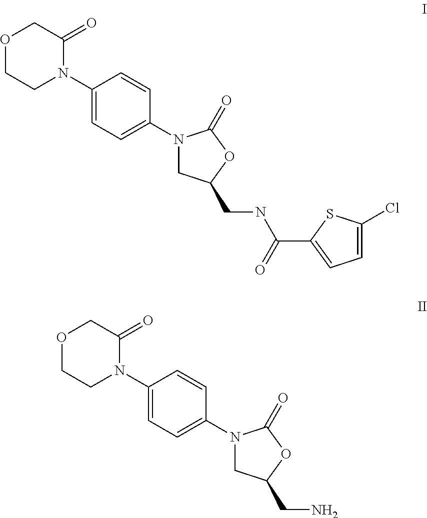

### BACKGROUND OF THE INVENTION

The compound 5-chloro-N-({(5S)-2-oxo-3-[4-(3-oxo-4-morpholinyl)phenyl]-1,3-oxazolidin-5-yl}methyl)-2-thiophenecarboxamide (I) was described for the first time in WO01/47919 (whose United States equivalent is United States Patent Application Publication No. 2010/137274; U.S. Pat. No. 7,157,456; U.S. Pat. No. 7,576,111; U.S. Pat. No. 7,585,860 and U.S. Pat. No. 7,592,339). For the synthesis, two alternative routes were disclosed therein. The first alternative starts from a chiral epoxide, which is opened nucleophilically by reaction with 4-(4-aminophenyl)-morpholin-3-one and is then cyclized with a phosgene equivalent (route B).

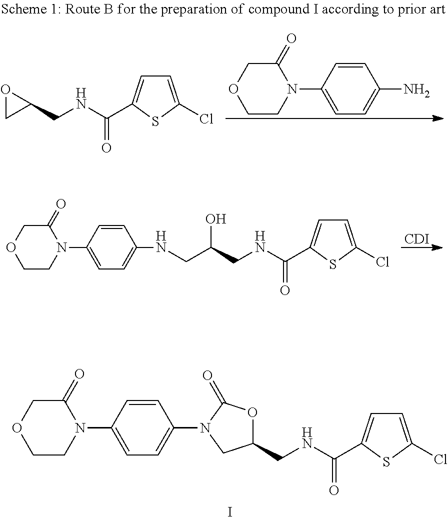

For the second alternative, the intermediate 4-[4-((S)-5-aminomethyl-2-oxooxazolidin-3-yl)phenyl]morpholin-3-one (II) is converted to the desired product by reaction with 5-chlorothiophene-2-carbonyl chloride or the product of the reaction of the free carboxylic acid with a combination of EDCI and HOBT (1-hydroxy-1H-benzotriazole, N′-(3-dimethylaminopropyl)-N-ethylcarbodiimide) (route A; the substituent “X” in the formula scheme corresponds to a leaving group such as chloride or to one formed with the mentioned typical activation reagents). The reaction here as per WO01/47919 takes place in an inert solvent in the presence of a base; in the examples, e.g. diisopropylethylamine is used.

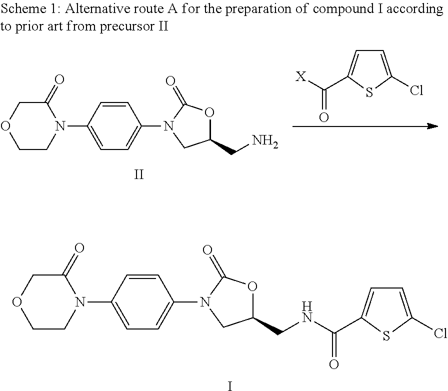

In WO05/068456, (whose United States equivalent is U.S. Pat. No. 7,351,823) an improvement in this synthesis is described, in which the reaction of 5-chlorothiophene-2-carbonyl chloride with the hydrochloride of intermediate II is carried out in the presence of an inorganic base in an ether, alcohol, ketone, in water or else in any desired mixture of these solvents. Moreover, further methods for the synthesis of 5-chloro-N-({(5S)-2-oxo-3-[4-(3-oxo-4-morpholinyl)phenyl]-1,3-oxazolidin-5-yl}methyl)-2-thiophenecarboxamide (I) have been described, although these do not pass through the intermediate 4-[4-((S)-5-aminomethyl-2-oxooxazolidin-3-yl)phenyl]morpholin-3-one (II) and therefore do not belong to the prior art, from which the present invention stands out.

The process described in WO01/47919 has the disadvantage that toxic reagents are used and an industrial conversion is difficult, while the improved process from WO05/068456 is based on 5-chlorothiophene-2-carbonyl chloride, which signifies an additional reaction step, namely the preparation of the acid chloride from the carboxylic acid. As a result, the process becomes unattractive compared with a direct access from 5-chlorothiophene-2-carboxylic acid. Moreover, for the chlorination of the carboxylic acid, extremely caustic and corrosive thionyl chloride is required. In order to achieve the purity necessary for use as drugs, the product of the reaction, moreover, has to be recrystallized from acetic acid.

### SUMMARY OF ADVANTAGEOUS EMBODIMENTS OF THE INVENTION

This gave rise to the problem of providing a process for the synthesis of 5-chloro-N-({(5S)-2-oxo-3-[4-(3-oxo-4-morpholinyl)phenyl]-1,3-oxazolidin-5-yl}methyl)-2-thiophenecarboxamide (I) from 4-[4-((S)-5-aminomethyl-2-oxooxazolidin-3-yl)phenyl]morpholin-3-one (II) and/or a suitable salt of the compound with a mineral acid or organic acid of the compound, in which 5-chlorothiophene-2-carboxylic acid can be used directly, which can be carried out on an industrial scale, dispenses with toxic solvents and reagents and produces the target product in high purity.

Surprisingly, it has now been found that the synthetic linking of the amide bond from 5-chlorothiophene-2-carboxylic acid and the primary amino function in the 4-[4-((S)-5-aminomethyl-2-oxooxazolidin-3-yl)phenyl]morpholin-3-one (II) can be carried out particularly well in the presence of propanephosphonic anhydride (T3P®). This is likewise the case when II is used as salt.

### DETAILED DESCRIPTION OF ADVANTAGEOUS EMBODIMENTS OF THE INVENTION

Accordingly, the invention provides a process for the reaction of 5-chlorothiophene-2-carboxylic acid with 4-[4-((S)-5-aminomethyl-2-oxooxazolidin-3-yl)phenyl]morpholin-3-one (II) and/or a suitable salt of the compound with a mineral acid or an organic acid to give 5-chloro-N-({(5S)-2-oxo-3-[4-(3-oxo-4-morpholinyl)phenyl]-1,3-oxazolidin-5-yl}methyl)-2-thiophenecarboxamide (I), which comprises carrying out the reaction in the presence of propanephosphonic anhydride (T3P®, CAS No. 68957-94-8). In this way, in just one step, a very clean product is obtained which can be further purified by an additional recrystallization from NMP (N-methylpyrrolidone) and in which all secondary components in the HPLC can be reduced to a level of in each case less than 0.03% (a/a).

Preference is given to the use of 5-chlorothiophene-2-carboxylic acid with 4-[4-((S)-5-aminomethyl-2-oxooxazolidin-3-yl)phenyl]morpholin-3-one (II) or 4-[4-((S)-5-aminomethyl-2-oxooxazolidin-3-yl)phenyl]morpholin-3-one as salt of an inorganic or organic acid, and particular preference is given to the use of the hydrochloride.

The coupling reagent propanephosphonic anhydride, of which preferably 0.8 to 2 equivalents are used, particularly preferably 1.2 to 1.8 equivalents, preferably used as ca. 50% strength solution, permits the desired reaction for the formation of the amide bond under very mild conditions, preferably in the presence of an organic base, which, in one particularly preferred embodiment of the invention, is triethylamine or ethyldiisopropylamine. In this connection, preference is given to using 1 to 8 equivalents of the base, particularly preferably 3 to 6 equivalents. Furthermore, the reaction can take place in a nontoxic medium, preferably in dialkyl ethers or carboxylic acid alkyl esters, particularly preferably in ethyl acetate.

The process according to the invention is also notable for the fact that the reaction can be carried out within a particularly mild temperature window, preferably between −20 and +80° C., particularly preferably between −10 and +50° C. and very particularly preferably between 0° C. and room temperature (+23° C.).

According to the invention, the work-up preferably takes place by adding water to the reaction mixture, whereupon the product is produced in the form of a solid and can thus be isolated in a simple manner.

The product thus obtained can be isolated as pure product for example by dissolving in hot organic solvent, preferably N-methylpyrrolidone or acetic acid, and subsequent crystallization by cooling the solution to preferably a temperature between 0° C. and +35° C. and preferably by adding pure 5-chloro-N-({(5S)-2-oxo-3-[4-(3-oxo-4-morpholinyl)phenyl]-1,3-oxazolidin-5-yl}methyl)-2-thiophenecarboxamide (I). The crystallization is particularly preferably from NMP, which produces a particularly pure product.

The invention is described in more detail below by a preferred embodiment, but is not limited to this:

### EXAMPLES

### Example 1

### Reaction of 5-chlorothiophene-2-carboxylic acid with 4-[4-((S)-4-aminomethyl-2-oxoimidazolidin-1-yl)phenyl]morpholin-3-one (II) to give 5-chloro-N-({(5S)-2-oxo-3-[4-(3-oxo-4-morpholinyl)phenyl]-1,3-oxazolidin-5-yl}methyl)-2-thiophenecarboxamide (I)

47.0 g of 4-[4-((S)-5-aminomethyl-2-oxooxazolidin-3-yl)phenyl]morpholin-3-one hydrochloride and 28.0 g of 5-chlorothiophene-2-carboxylic acid are introduced as initial charge together with 75.0 g of ethyldiisopropylamine in 132 g of ethyl acetate and cooled to 10° C. 125 g of a 50% strength solution of propanephosphonic anhydride (T3P®) in ethyl acetate are then metered in over a period of ca. 30 minutes. During this, the temperature is kept at 10-15° C. The mixture is further stirred for ca. 12 h and during this time the temperature is slowly increased to 25° C. 210 g of water are then added dropwise over a period of ca. 60 minutes. The multiphase mixture is stirred for a further 60 minutes and then filtered with suction. The crude product is washed with 100 g of water and then with 100 g of acetone and then dried. This gives 58.9 g (94% of theory) of 5-chloro-N-({(5S)-2-oxo-3-[4-(3-oxo-4-morpholinyl)phenyl]-1,3-oxazolidin-5-yl}methyl)-2-thiophenecarboxamide (I). The purity of the product obtained in this way is >98% area percent (HPLC).

### Example 2

### Recrystallization of 5-chloro-N-({(5S)-2-oxo-3-[4-(3-oxo-4-morpholinyl)phenyl]-1,3-oxazolidin-5-yl}methyl)-2-thiophenecarboxamide (I)

60.0 g of the product obtained according to example 1 are suspended in 300 g of NMP and the mixture is heated to 100° C. The mixture is stirred for 10 to 20 minutes and then slowly cooled to room temperature over a period of 1.5 hours. The mixture is then further stirred for a period of minutes and then the product is filtered off with suction. The filter cake is washed with 300 g of water and then dried to constant weight. This gives 54.3 g of product with a purity of 99.9% area percent (HPLC), the largest individual impurity after reworking is less than 0.05% (HPLC, a/a).

This corresponds to 87% of theory (total over reaction and recrystallization) and 92% recrystallization yield.

1. A process for the preparation of 5-chloro-N-({(5S)-2-oxo-3-[4-(3-oxo-4-morpholinyl)phenyl]-1,3-oxazolidin-5-yl}methyl)-2-thiophenecarboxamide (I) comprising reacting 5-chlorothiophene-2-carboxylic acid and 4-[4-((S)-5-aminomethyl-2-oxooxazolidin-3-yl)phenyl]morpholin-3-one (II) 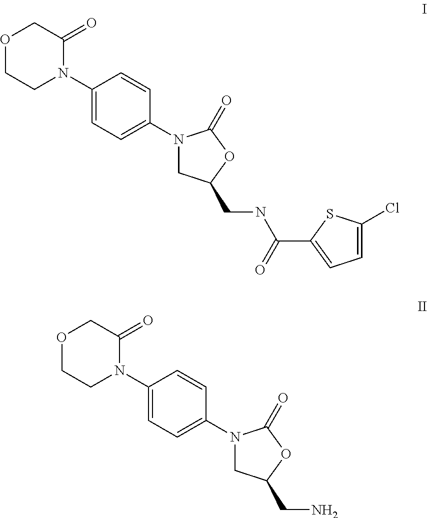
or a salt of this compound with a mineral acid or an organic acid in the presence of propanephosphonic anhydride.

2. The process as claimed in claim 1, wherein the reaction is carried out in the presence of an organic base.

3. The process as claimed in claim 1, wherein the reaction is carried out in the presence of a tertiary amine.

4. The process as claimed in claim 1, wherein the reaction is carried out in the presence of triethylamine, N-methylmorpholine or ethyldiisopropylamine.

5. The process as claimed in claim 1, wherein 5-chlorothiophene-2-carboxylic acid is reacted with 4-[4-((S)-5-aminomethyl-2-oxooxazolidin-3-yl)phenyl]morpholin-3-one (II).

6. The process as claimed in claim 1, wherein 5-chlorothiophene-2-carboxylic acid is reacted with 4-[4-((S)-5-aminomethyl-2-oxooxazolidin-3-yl)phenyl]morpholin-3-one hydrochloride.

7. The process as claimed in claim 1, wherein the reaction is carried out at a temperature between −20 and +50° C.

8. The process as claimed in claim 1, wherein the reaction is carried out in an organic solvent selected from dialkyl ethers or carboxylic acid alkyl esters.

9. The process as claimed in claim 1, wherein the reaction is carried out in ethyl acetate.

10. The process as claimed in claim 1, wherein the propanephosphonic anhydride is in solution in ethyl acetate.

11. The process as claimed in claim 1, wherein the reaction product is recrystallized for purification from NMP or a mixture of NMP with any desired other solvent with an NMP fraction of more than 60%.
